(19) United States
(12) Patent Application Publication
(10) Pub. No.: US 2019/0198162 A1
(43) Pub. Date: Jun. 27, 2019

(54) COMPUTERIZED HOSPITAL RAPID STAFFING PROCESS

(71) Applicant: Sally F. Nuñez, New York, NY (US)

(72) Inventor: Sally F. Nuñez, New York, NY (US)

(21) Appl. No.: 16/231,826

(22) Filed: Dec. 24, 2018

Related U.S. Application Data
(60) Provisional application No. 62/609,533, filed on Dec. 22, 2017.

(51) Int. Cl. G16H 40/20; G06Q 10/06; G06Q 50/22

(52) U.S. Cl. CPC G16H 40/20; G06Q 50/22; G06Q 10/063118

(57) ABSTRACT

A computerized process includes, within a computerized server device, operating programming configured to receive a request to fill a medical staff shift position, determine a list of necessary qualifications required to fill the medical staff shift position based upon the request, determine an urgency level of the request, publish the request to one of a staffing agency and a plurality of candidate staff members based upon the determined list and the determined urgency level, monitor a response to the published request, fill the medical staff shift position with an elected staff member based upon the monitored responses, and publish the elected staff member. The process can further include automatically select an elected staff member to fill the medical staff shift position.

### CROSS REFERENCE TO RELATED APPLICATIONS

This disclosure claims the benefit of U.S. Provisional Patent Application No. 62/609,533 filed on Dec. 22, 2017, which is hereby incorporated by reference.

### TECHNICAL FIELD

This disclosure is related to a computerized process to staff a medical facility, and in particular, to enable the medical facility to post shift openings on short notice.

### BACKGROUND

The statements in this section merely provide background information related to the present disclosure. Accordingly, such statements are not intended to constitute an admission of prior art.

Computerized systems are known for posting job openings. In one example, an employer develops a description and a list of requirements for the job opening. Potential candidates can view the posted opening and reply through provided means. In another example, candidates are invited to post resumes in a database, and employers can search through the database.

Hospitals and other medical facilities can keep lists of temporary or contract basis individuals that can be used during staffing shortages or medical emergencies to fill emergency openings. However, known methods create confusion, with mass emails or automated phone messages generating a wave of replies that need to be sifted through.

Homecare agencies fill requests for in home medical care and can keep lists of temporary or contract basis individuals that can be used to fill openings to provide in home care. However, known methods create similarly confusion, with mass emails or automated phone messages generating a wave of replies that need to be sifted through.

### SUMMARY

A computerized process includes, within a computerized server device, operating programming configured to receive a request to fill a medical staff shift position, determine a list of necessary qualifications required to fill the medical staff shift position based upon the request, determine an urgency level of the request, publish the request to one of a staffing agency and a plurality of candidate staff members based upon the determined list and the determined urgency level, monitor a response to the published request, fill the medical staff shift position with an elected staff member based upon the monitored responses, and publish the elected staff member. The process can further include automatically select an elected staff member to fill the medical staff shift position.

### BRIEF DESCRIPTION OF THE DRAWINGS

One or more embodiments will now be described, by way of example, with reference to the accompanying drawings, in which:

FIG. 1 illustrates operation of an exemplary server device configured to run a medical staff position filling process, in accordance with the present disclosure;

FIG. 2 illustrates an exemplary computerized cellular phone configured to run a portion of the disclosed process for a hospital administrator, in accordance with the present disclosure;

FIG. 3 illustrates operation of an exemplary server device configured to run a homecare position filling process, in accordance with the present disclosure;

FIG. 4 illustrates through a flow chart steps of a computerized medical staff position filling process, in accordance with the present disclosure;

FIG. 5 illustrates through a flow chart steps of a computerized homecare position filling process, in accordance with the present disclosure; and

FIG. 6 illustrates through a flow chart steps of automatically generating requests to fill medical staff shift positions based upon monitoring requests for vacation and call out sick days in a facility, in accordance with the present disclosure.

### DETAILED DESCRIPTION

A computerized process is provided, permitting a hospital or similar medical facility to quickly post temporary, short-notice, or emergency shift postings. The process is provided to enable the medical facility to request for a staff on short notice and able to request for a new candidate. These requests are claimed by qualified candidates and in alternative, an agency administrator can assign these candidates. In addition or in the alternative, the disclosed process can be used to fill requests for homecare with medical professionals.

The disclosed system and process can be used for businesses that need staffing (cleaning agency, delivery agencies, etc.) as an alternative to using traditional systems that use optimization techniques to organize work schedule of labor based types of work. The system and process can also be applied to individual consumers for providing home-based healthcare delivery (Telehealth) where private individuals can request private care services at the comfort of their home for themselves or for their loved ones.

The disclosed system is discussed throughout as being useful for hospitals. However, one will realize that the disclosed system can be used for large doctors' offices, nursing homes, natural disaster sites or emergency response sites, large public gatherings, large sporting events, or any other situation which can require medical staff on short notice. In another embodiment, the disclosed system can be useful to staff homecare networks, with medical staff being scheduled to provide in house care for elderly or infirmed patients.

One embodiment of the process or computerized application disclosed is provided with a name “BackApp.” BackApp originated from the word “Back-Up” which means “to support”. It is a 24/7 staffing solution. Since staffing is one of the major concerns of most facilities, an application was developed with a goal to simplify and address the difficulty of staffing in the facility.

This computerized application streamlines the fulfillment process of staffing needs by sending the request in just a click and receives confirmation in minutes. This is a one-stop marketplace for all the staffing needs.

In one embodiment, BackApp can be downloaded using Google Play® and the Apple AppStore®. BackApp provides training to all users regarding the basics of the system and what services are provided. BackApp provides 24/7 technical support.

Two exemplary uses of the disclosed system are detailed. A first is an urgent need request. An urgent need request is a need of staff for an upcoming shift, for example, a need for a staff before a two hour window prior to the shift or within the two hour window. Any urgent timing window can be defined, including but not limited to one hour, two hours, four hours, eight hours, and a day. The system can quickly put out an urgent need request which can go in series or in parallel to an identified list of staff candidates and a staffing agency. Benefits of this system are avoiding a flurry of responses which the hospital staff must sift through or paying for expensive overtime, simply carrying medical staff over from one shift to the next.

A second use of the disclosed system is a new staff request. A new staff request is used to request for a new staff for a full-time position or per diem position. The facility can send a request to the agency to fill in a vacant position or add an additional staff using the disclosed system. According to one embodiment, a difference of this request to an urgent need request can include that the facility must add the interview date and the orientation date. In this case, a staffing agency's recruitment process will be based on the interview and orientation date given by the facility.

In one embodiment, a hospital can operate the disclosed system, make contractual agreements with a staffing agency, and the agency can refer candidate medical staff to a cellular phone application to register their names, qualifications, work history, licensure, etc. such that as requests to fill positions come through, the candidates can be automatically filled or polled to fill the requests. In another embodiment, the hospital can operate the disclosed system and provide or refer the disclosed system as a website or cellular application to every candidate that contacts the hospital for work opportunities. In another embodiment, the staffing agency can run the disclosed system publish availability of the system to both hospitals and candidate staff, and act as a matching service, with the disclosed system automatically filling requests to fill positions with qualified candidates.

In one embodiment a critical two hour window prior to the start of a work shift at a medical facility can be defined. Prior to two hours before the shift start, one can say that there is adequate time to fill the shift through non-urgent means. However, within the two hour window prior to the start of a shift, a new request for staff to fill an open position can be deemed urgent, with a serious risk that paperwork and administrative delays can prevent a qualified candidate being found to fill the open position. The disclosed system can be used even if the need to fill the open position is urgent. By automatically notifying candidate staff of the open position, receiving replies from the candidate staff, and automatically selecting between the responding candidate staff to name an elected staff to fill the position, administrative delays and paperwork can be avoided and the urgent need for medical staff can be filled. Further, by removing the staffing concerns of clinicians, the disclosed system reduces the burden of administration and permits the physicians to focus more time on patient care.

The disclosed system can be an open registration system, permitting new candidate workers to learn of the system, download the necessary cellular application or access the portal website, and enter all necessary information to register a candidate staff member. In another embodiment, hospitals or a staffing agency can pre-screen potential candidate staff, with hospital administration interviewing and approving each member of a pool of candidate staff. The disclosed system can automatically screen potential candidate staff, with certain levels of licensure and training required for a person to be considered a candidate staff member for certain open positions. The disclosed system can aid in getting candidate staffs credentials verified, for example, with the system prompting state licensure boards to verify the identity and license status for certain candidates. For required training, the system can automatically prompt training facilities to verify the identity and status of the candidate having completed certain training. In another embodiment, the disclosed system can have access to online training materials and provide an opportunity for candidates to complete training through the system. For example, a City Hospital can have certain site specific procedures that they want followed. Through the system, City Hospital can make available a training video and related confirmation test for candidates to take, after which they are certified as having been trained in that site's procedures. Such training certification can be managed, for example, expiring if the candidate does not work at that facility for more than an exemplary three month period.

Exemplary operation of the computerized process provides a hospital management portal, such as through a desktop computer or a mobile phone, wherein a hospital administrator may designate shift postings that need to be filled. The management portal can provide the administrator with a variety of options, for example, enabling the administrator to select exemplary minimum licensing, training, and pre-determined person specific ratings for each shift posting. The administrator can select pay levels for the postings, for example, with options to select normal pay, perked pay scaled to attract candidates during a time of particular shortages, and emergency pay when skilled candidates are urgently needed. In another embodiment, the system can be integrated with a payroll or human resources system, wherein all shifts and anomalies such as vacations, sick days, etc. for the facility are managed, and when open positions are evident as a result of those automatically managed anomalies, the system automatically generates a request to fill the position in accordance with this disclosure.

The disclosed system can include a shift management or shift vacancy function. In one embodiment, a scheduler process can be implemented wherein the disclosed system can embody programming to schedule all work shifts for every employee of an exemplary hospital, with a staffing coordinator at the hospital entering and managing employee data for every shift for every position in the hospital. The system could then manage vacation days and sick days for all employees, for example, with the employees being provided an automated phone system or a web-based system in which to request vacation days or to call in sick. The system would then be able to identify unfilled shifts and create a request in accordance with this disclosure to fill the open shift.

The disclosed system can further include a payroll function, which can include on-site card scanning devices or punch-in punch-out clocks, with employees scheduled through the request system being able to directly log their time through the scanning device or punch-in punch-out clock. The disclosed system can then implement programming to request a check to pay the worker or to directly generate and automatically send the check or wire transfer payment.

The disclosed system can include a human resources function, for example, requiring each new candidate staff member to electronically submit details about a professional license and completed training. In one embodiment, the system can automatically track expiration dates for licenses, training, and continuing education requirements and provide automatic reminders to registered workers.

The disclosed system can include an automatic invoicing function, with automatic invoices showing amounts due being generated and sent to either facilities making use of the service or patients that submit requests.

The disclosed system can automatically generate an administrative report to facility management, for example, including weekly or monthly reporting to administration how many shifts were filled, the names of staff that filled the shifts, the ratings that the physicians or other staff provided the elected staff members that filled the roles, the total cost of the elected staff members that were used, etc.

Candidates for the positions can register with the hospital or medical facilities to receive notifications of job postings fitting certain criteria. For example, a nurse with a day shift job four days a week at one hospital may ask to receive electronic notifications on his or her phone when the hospital running the process posts a perked pay or emergency pay posting on the three days that he or she does not work in their first job. A nurses aid with children can ask only for postings that include at least 24 hours notice so that he or she can arrange for child care.

A wide array of license, training, and pre-determined person specific criteria can be used to automatically or semi-automatically fill candidates into roles. Hospital administration can manage pre-determined ratings for each candidate, for example, based upon past performance at the hospital, interview scores, reliability, etc. For example, a posting may require that a registered nurse (RN), with trauma level one training, with at least an 85/100 pre-determined rating for that hospital be required to fill a particular posting. In one example, with a normal priority for the posting, any candidates that do not fit those criteria can be filtered out, and a top candidate can be selected based upon comparison of the pre-determined rating values for each qualifying candidate. Such a pre-determined rating can, for example, be a composite score for a person, such as ratings provided by facility physicians and administrators, track records of being available and arriving to work on time, and years of service in the profession.

Hospitals can have floating in-house staff available. According to one embodiment, the system can include a list of floating staff that are contacted first or in parallel to candidate staff members and given preferential scheduling over the candidate staff members.

In another example, a posting can be filled based upon a best qualified candidate when a perfectly qualified candidate is not available. For example, in an emergency, the process can select a candidate based upon a fewest number of deviations from the qualifications listed for a posting, and the candidate and a description of the deviations can be supplied to the hospital administrator for approval or dis-approval. In one example where the hospital administrator does not approve of the deviation, the administrator can be given the option to re-post the job with a different pay designation, for example, advancing the pay from normal to a perked level. In another example, the system can automatically use market-driven forces to fill positions, with open positions being initially offered with some base or minimum pay, and with automatic re-postings of the open positions incrementally increasing the offered rate until the open positions are filled. Advantages of the system and process can include automatic checking of licensing of candidates, integrated tracking of hospital specific training courses offered to candidates registered in the system, and automatic execution of ratings generating polls, for example, asking physicians by email to rate candidates that filled a role in a shift with the physician. Further, pay can be according to set scales. In another example, pay can adjust automatically, for example, based upon gathered information such as the pay levels offered by competing facilities, scarcity of candidates on a certain day or with certain qualifications, and other factors. In one embodiment, the administrator can be presented with a recommended pay level for the posting, and the administrator can use a graphic interface such as a dial graphic to adjust the desired pay level for the posting according to a desired result. The system can select qualified candidates from pool of staff expressing interest in fulfilling a request to fill an open position. In other embodiments, selection of the elected staff to fill the open position can be made by a first qualified candidate staff member claiming the request, a staffing agency or designated hospital administrator manually assigning a specific staff from a list of candidate staff, or by the system automatically assigning candidate staff to a request by some hybrid model, for example, with a request being entered with a bias to select staff already associated with the facility before filling the role with another qualified candidate not already associated with the facility.

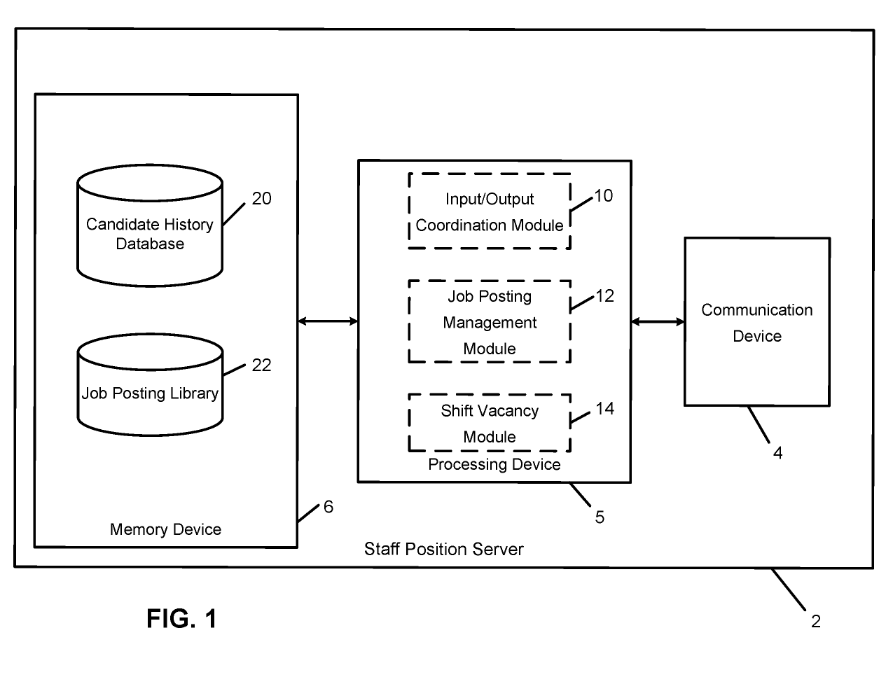

Referring now to the drawings, wherein the showings are for the purpose of illustrating certain exemplary embodiments only and not for the purpose of limiting the same, FIG. 1 illustrates operation of an exemplary server device configured to run a medical staff position filling process. Server device 2 is illustrated, including a device configured to remotely provide Internet functionality to a plurality of users connected remotely to the server device. The server device includes a computerized processor, random access memory (RAM), and a durable memory device. Server 2 is illustrated, including a communications device 4 configured to connect to users through the Internet or similar network, processing device 5 configured to operate programming configured to execute program steps, and memory device 6 capable of storing libraries or databases of information that can be retrieved for use in the programs of processing device 5.

Processing device 5 can operated computerized modules or units configured to execute process steps or discrete operations. Exemplary modules are provided. It will be understood that a wide variety of programming techniques and options can be used to achieve particular results or output from the system, and the disclosure is not intended to be limited by the exemplary module descriptions provided herein. An exemplary input/output coordination module 10 is illustrated configured to receive inputs from hospital administration, candidates for postings, physicians, and other users that may make use of the application. An exemplary job posting management module 12 is provided, including programming configured to create, manage, and fill job postings based upon input from the hospital administrator. An optional shift vacancy module 14 is illustrated which receives information from a payroll or vacation scheduling system with details regarding shifts to be worked in a medical facility, wherein shift vacancy module 14 operates programming to automatically identify open medical staff shift positions that need to be filled to automatically generate requests to fill the open medical staff shift positions.

Memory device 6 includes a candidate history database 20 and a job posting library 22. Candidate history database 20 includes information on all of the candidates, for example, for a particular hospital, including but not limited to their contact information, their licensing levels, their completed training certificates, and ratings information. Job posting library 22 includes pre-saved and editable templates for job postings that can selected, populated, and published by a hospital administrator.

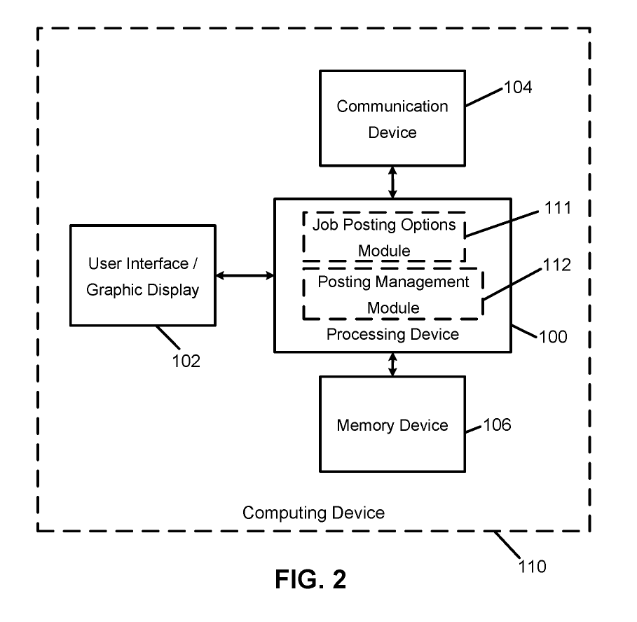

FIG. 2 illustrates an exemplary computerized cellular phone configured to run a portion of the disclosed process for a hospital administrator. Exemplary cellular device 110, embodied as a typical smart phone, is illustrated, including a device configured to connect with the BackApp server of FIG. 1 and coordinate data entry and output by a hospital administrator. It will be appreciated that a similar device with different modules can be used by candidates for the job postings. The cellular device 110 includes a computerized processor, random access memory (RAM), and a durable memory device. Cellular device 110 is illustrated, including a communications device 104 configured to connect to the server through the Internet or similar network, processing device 100 configured to operate programming configured to execute program steps, memory device 106 capable of storing libraries or databases of information that can be retrieved for use in the programs of processing device 100, and user interface/graphic display 102.

Processing device 100 includes two exemplary modules operating programming. Job postings options module 111 provides the hospital administrator with a template and selectable options to configure a job posting or multiple job postings to be published. Processor 100 can optionally be also connected with a hospital specific server, for example, so that open postings can be automatically transferred over from a staffing program operated by the hospital (already tracking employee filled shifts at the hospital and identifying unfilled shifts.) Posting management module 112 coordinates information related to candidates automatically filled by the server into certain postings. Module 112 can inform the administrator of the filled postings. In another embodiment, module 112 can present a proposed roster of candidates filling roles, and the administrator can approve or disapprove of each proposed candidate. If a candidate is disapproved, the administrator can manually adjust the postings or can order that the server refill the positions based upon the disqualified or demoted candidate.

Memory device 106 can include information about the postings, favorite settings for the administrator for past postings, hospital specific pay information for use by the administrator, for example, tracking a staffing budget for a particular time period, and personalized notes and reminders about candidates.

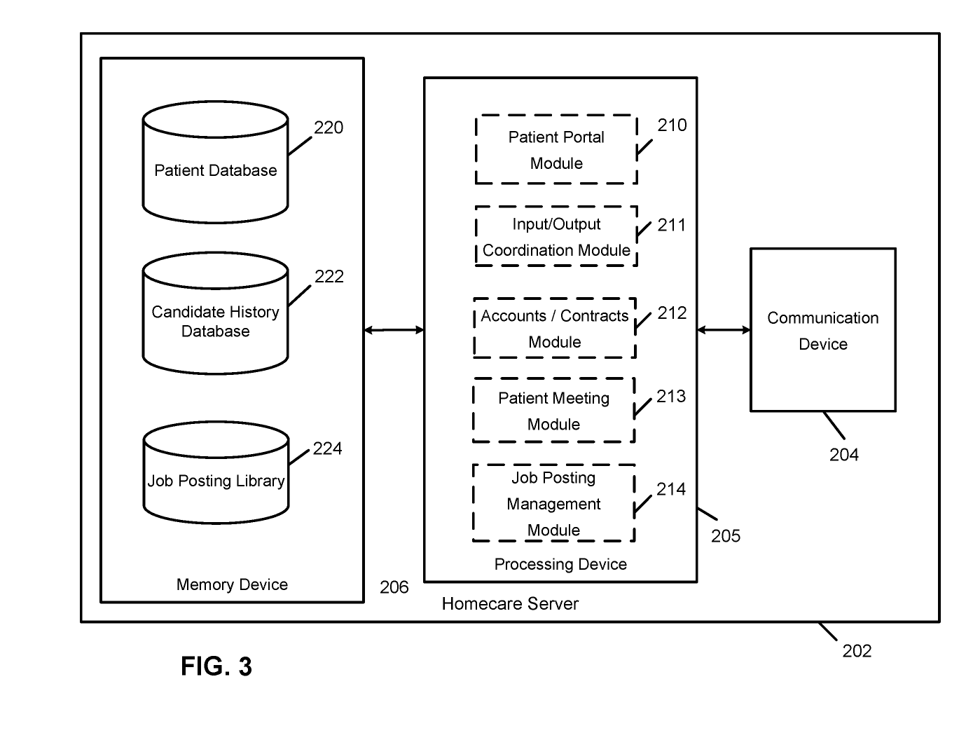

FIG. 3 illustrates operation of an exemplary server device configured to run a homecare position filling process. Server device 202 is illustrated, including a device configured to remotely provide Internet functionality to a plurality of users connected remotely to the server device. The server device includes a computerized processor, random access memory (RAM), and a durable memory device. Server 202 is illustrated, including a communications device 204 configured to connect to users through the Internet or similar network, processing device 205 configured to operate programming configured to execute program steps, and memory device 206 capable of storing libraries or databases of information that can be retrieved for use in the programs of processing device 205.

Processing device 205 can operated computerized modules or units configured to execute process steps or discrete operations. Exemplary modules are provided. It will be understood that a wide variety of programming techniques and options can be used to achieve particular results or output from the system, and the disclosure is not intended to be limited by the exemplary module descriptions provided herein. An exemplary patient portal module 210 is illustrated configured to present request details for selection and payment entry screens remotely to patients logging into the system. An exemplary input/output coordination module 211 is illustrated configured to receive inputs from a staffing agency operating the homecare network, candidates for postings, nurses, caregivers, and other users that may make use of the application. An exemplary accounts and contracts module is illustrated configured to manage patient accounts, billing agreements, and payment processing. An exemplary patient meeting module 213 is illustrated configured to manage and schedule events where a case manager and a patient meet either in person or telephonically to arrange details regarding care to be provided. An exemplary job posting management module 214 is provided, including programming configured to create, manage, and fill job postings based upon input from the staffing agency.

Memory device 206 includes a patient database 220, a candidate history database 222, and a job posting library 224. Patient database 220 includes information, personal data, billing records, treatment details, etc. for each patient registered to the system. Candidate history database 222 includes information on all of the candidates for a homecare network, including but not limited to their contact information, their licensing levels, their completed training certificates, and ratings information. Job posting library 224 includes pre-saved and editable templates for job postings that can selected, populated, and published by a staffing agency managing the homecare network.

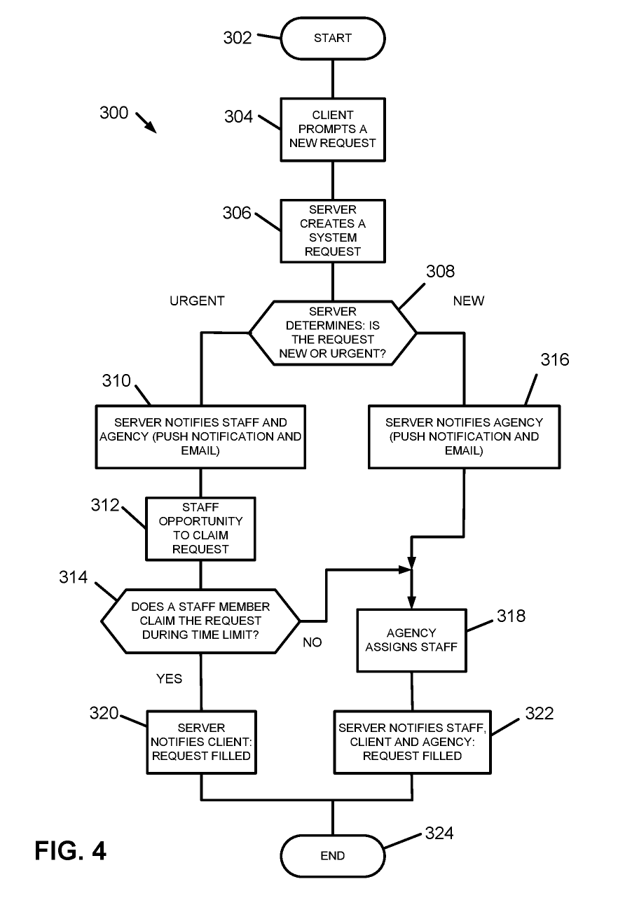

FIG. 4 illustrates through a flow chart steps of a computerized medical staff position filling process. A number of different exemplary processes for filling medical staff positions in accordance with the present disclosure are envisioned. A non-limiting exemplary process 300 is provided illustrating a process for filling open medical staff shift positions in a medical facility. Process 300 starts at step 302. At step 304, a client, such as a hospital or a staffing agency contracted to provide temporary staff at the hospital, prompts or enters through an exemplary web-based request screen a request to fill an open medical staff position. At step 306, a remote server device in communication with the web-based request screen creates a system request. The system request can include information such as the medical facility for the open position, the department within the facility with the open positions, start and stop times for the shift to be worked, pay offered for filling the open position, qualifications required to fill the open position, and optional criteria that the user can select for prioritizing candidates to fill the position. At step 308, the server determines whether the request is a new request or an urgent request. A new request can be a open position for one shift at some time in the future not within a defined urgent timing window, or a new request can be an open position on a temporary but repeating basis, a part time permanent basis, or a full time permanent basis. An urgent request can be an open position for one shift, wherein the current time is within a urgent timing window, for example, within a specified number of hours of the start of the open position shift. In another example, an urgent request can be an open position for a medical staff position that has been historically difficult to fill, for instance, for a medical professional with rare training. If the request is determined by the server to be urgent, the process advances to step 310; if the request is determined to be new, the process advances to step 316. At step 310, the request being urgent and in need of immediate review by as many potential candidates to fill the position as possible, the server notifies a list of candidate staff members that are registered with the system of the request and provides the members the opportunity to directly respond to the request to the server. In one embodiment, in parallel to the notification sent directly to the members, a notification can also be sent to staffing agencies to give them the opportunity to respond to the request in parallel. At step 312, the candidate staff members respond to the request, with some portion of the members attempting to claim the open position in the request. At step 314, the server determines whether at least one qualified candidate staff member has claimed the request during a specified time limit. If more than one qualified member has attempted to claim the open position, then the server can prioritize/rank/score the candidates to select and elected staff member to fill the position. A time limit to accept the request can be defined, for example, with one hour prior to the shift starting being an exemplary expiration of the time limit. Once the time limit has expired, the request can be deemed an emergency, the process can advance to step 318, where a staffing agency can be contacted to immediately fill the position. If the position is filled at step 314, the process advances to step 320 where the server notifies the client that the position has been filled and the request can be closed.

For new requests, at step 316, the server notifies a staffing agency or agencies of the request for the open medical staff shift position to be filled. At step 318, the agency responds to the request, providing a candidate staff member or an elected staff member to fill the position. In one embodiment, if several agencies are used in parallel, the server can prioritize/rank/score the candidate members provided by the various agencies and select one as an elected staff member. In other embodiments, where the agency has been delegated the role of selecting an elected staff member for open positions, the agency responds at step 318 with the elected staff member. At step 322, the server notifies the medical staff, the client, and the agency that the request is filled and closed. At step 324, the process ends.

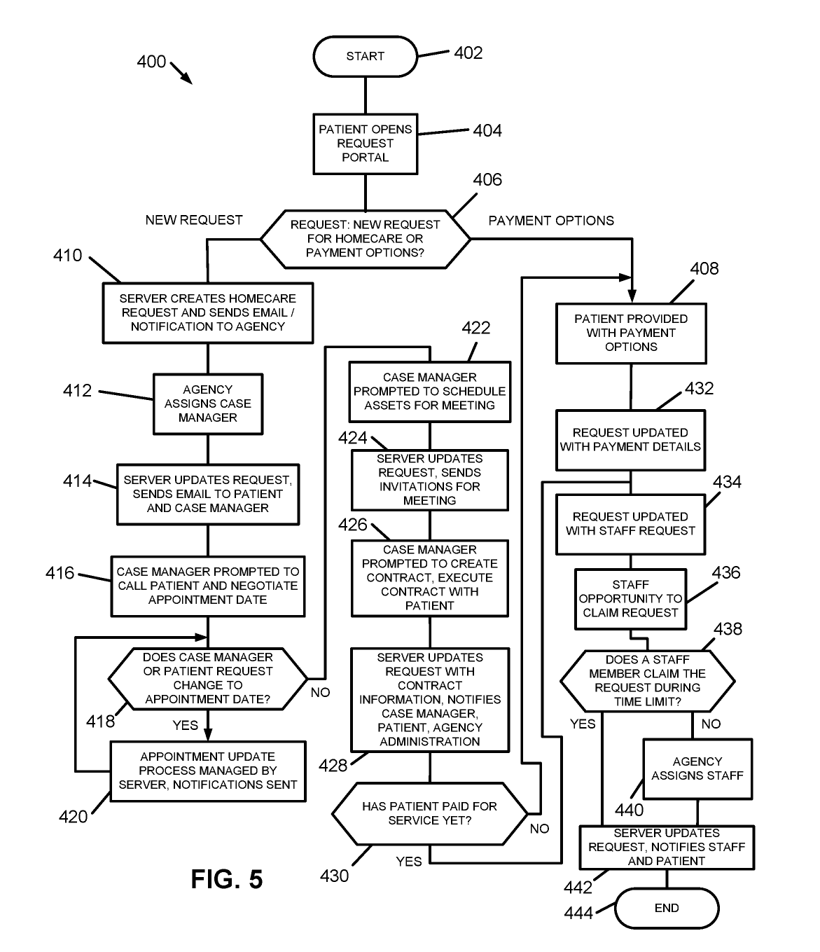

FIG. 5 illustrates through a flow chart steps of a computerized homecare position filling process. A number of different exemplary processes for filling medical staff positions in accordance with the present disclosure are envisioned. A non-limiting exemplary process 400 is provided illustrating a process for filling open medical staff shift positions in a homecare network. Process 400 starts at step 402. At step 404, a patient opens a request portal, for example, through a cellular device application. The portal can offer an ability for the patient to enter details for a request to fill an open medical staff shift position. Such details can include name of the patient, address of the patient, contact information for the patient, dates and times for shifts that need to be filled, medical history of the patient, contact information for a primary care physician for the patient, insurance information, etc. The portal can also offer an ability for the patient to provide payment for services. At step 406, the server determines whether the patient has entered a new request or has requested to make a payment. If the patient has requested to make a payment, then the process advances to step 408, where the patient is presented with payment options. If the patient has entered a new request to fill an open medical staff shift position or positions, the process advances to step 410. At step 410, the server creates a homecare system request and sends notification to a staffing agency. At step 412, the staffing agency responds to the request by providing a name and information for an elected case manager. At step 414, the server updates the request and sends communications to the patient and the elected case manager making an introduction. At step 416, the system prompts the case manager to call the patient and set up details for providing care. In one exemplary embodiment, details can be handled online through exchanges of forms to be filled out. In another embodiment, an appointment can be set up for the case manager, the patient, and any required resources for the meeting to meet, either in person, over the phone, or through other communications means, to arrange the details of providing homecare. At step 418, the server monitors and determines whether either the case manager or the patient request to change the appointment date. If so, the process advances to step 420 where the server facilitates the required parties finding a new date for an appointment. If no request to change the appointment date is received at step 418, the process advances to step 422, wherein the server prompts the case manager to schedule assets for the meeting. Assets for the meeting can include a candidate staff member to fill the position, an elected staff member that has been selected to fill the position, a representative from the patient's physician's office, a representative agent from the patient's insurance company, electronic resources for facilitating the meeting such as a call in number, and a physical meeting location for a meeting. At step 424, the server updates the request with confirmed resources for the meeting and sends out invitations to all of the parties involved. At step 426, the case manager is prompted to create a service contract and execute the contract with the patient. The system can provide templates or other assistance to the case manager. The system can include an option or requirement to upload the executed contract. The system can include e-signing or electronically-signing options, with the actual execution of the contract occurring through the server. At step 428, the system updates the request with details from the executed contract. At step 430, the server determines whether the patient has paid for services yet. If the patient has not yet paid, the process advances to step 408, wherein the patient is provided with payment options. When payment is made at step 408, the process advances to step 432 where the request is updated with details of the payment. If, at step 430, the patient has already paid, the process advances to step 434, where, if no elected staff member has yet been chosen, the request is sent out to candidate staff members. At step 436, responses from the candidate staff members are received. At step 438, it is determined whether at least one qualified candidate staff member has responded to the request. If not, at step 440, a staffing agency is contacted to fill the open position. If, at step 438, at least one qualified candidate staff member has responded, the server prioritizes/ranks/scores the candidates to select and elected staff member(s) to fill the position. At step 442, the server updates the request with an elected staff member(s) and notifies the elected staff member(s) and patient with relevant details regarding the filled request. At step 444, the process ends.

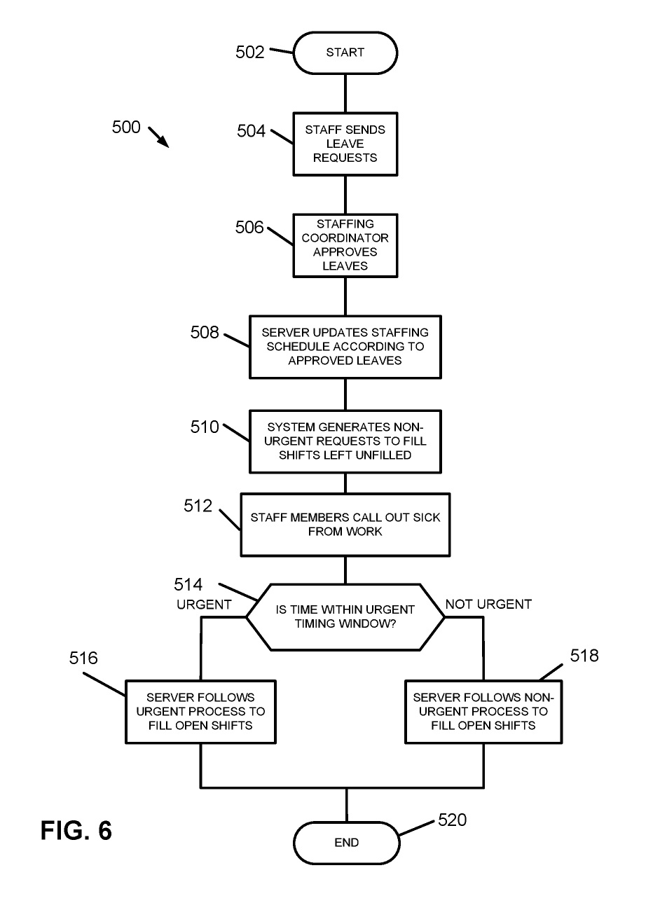

FIG. 6 illustrates through a flow chart steps of automatically generating requests to fill medical staff shift positions based upon monitoring requests for vacation and call out sick days in a facility. A number of different exemplary processes for filling medical staff positions in accordance with the present disclosure are envisioned. A non-limiting exemplary process 500 is provided illustrating a process for automatically generating requests to fill open medical shift positions and filling the open medical staff shift positions in a medical facility. Process 500 starts at step 502. At step 504, at least one staff member at the facility sends a vacation leave request. At step 506, the staffing coordinator approves the leave request. At step 508, the server updates a staffing schedule for the facility according to the approved leave. The server can include programming to analyze the staffing schedule and identify uncovered, unfilled, or open shifts. At step 510, the system generates a non-urgent requests to fill open or unfilled shifts in the staffing schedule. At step 512, at least one staff member of the facility calls out sick from work. At step 514, the server determines whether a time left to fill an open shift is within a defined urgent timing window for that shift. If it is within the urgent timing window, the process advances to step 516 where the server follows an urgent process, as described herein, to fill the open shift. If it is not within the urgent timing window, the process advances to step 518 where the server follows a non-urgent process, as described herein (for example, as a new request) to fill the open shift. At step 520, the process ends.

A number of methods to prioritize, score, or rank a plurality of candidate staff members are envisioned. According to one embodiment, a facility or network can interview all candidates, and the facility or network administration can maintain a candidate specific score for each candidate. It can be up to that administration to decide whether to update the candidate specific score as the candidate accrues time worked at the facility or in the network. Such a candidate specific score can generate or influence the overall score of the candidate. In another embodiment, years of experience in the medical field, in a particular licensed position, and/or time worked at a particular facility or in a particular network can generate or influence the overall score of the candidate. In another embodiment, certification of licenses for a candidate and required training for the candidate can be used to block the candidate from applying or responding to certain requests. For example, if the open position requires a licensed nurse practitioner to fill the open position, a candidate that has not registered such a license with the system will not be offered and will not be able to respond to such requests.

The generated requests can be sent out as individual offers, for example, within emails to each member of a qualified staff member list. In additional or the alternative, open requests can be searchable, with a candidate being able to view open requests. In one embodiment, a candidate will be able to see open requests that he/she is not yet qualified for, so as to encourage the candidate to complete required training and/or get licensing already in place certified within the system to enable the candidate to respond to those requests in the future.

The disclosure has described certain preferred embodiments and modifications of those embodiments. Further modifications and alterations may occur to others upon reading and understanding the specification. Therefore, it is intended that the disclosure not be limited to the particular embodiment(s) disclosed as the best mode contemplated for carrying out this disclosure, but that the disclosure will include all embodiments falling within the scope of the appended claims.

1. A computerized process, comprising:
within a computerized server device, operating programming configured to:
receive a request to fill a medical staff shift position;
determine a list of necessary qualifications required to fill the medical staff shift position based upon the request;
determine an urgency level of the request;
publish the request to one of a staffing agency and a plurality of candidate staff members based upon the determined list and the determined urgency level;
monitor a response to the published request;
fill the medical staff shift position with an elected staff member based upon the monitored responses; and
publish the elected staff member.

2. A computerized process, comprising:
within a computerized server device, operating programming configured to:
receive a request to fill a medical staff shift position;
determine a list of necessary qualifications required to fill the medical staff shift position based upon the request;
determine an urgency level of the request;
publish the request to a plurality of candidate staff members based upon the determined list and the determined urgency level;
monitor responses to the published request;
automatically select an elected staff member to fill the medical staff shift position from the monitored responses; and
publish the elected staff member.

3. The computerized process of claim 2, wherein the medical staff shift position comprises a homecare position requiring the elected staff member to provide in homecare to a patient.

4. The computerized process of claim 2, wherein the medical staff shift position comprises a position at a medical facility.

5. The computerized process of claim 2, wherein the list of necessary qualifications comprises at least one qualification selected from a licensure, a required training certification, a pre-determined hospital specific rating level, and a required pay level.

6. The computerized process of claim 2, wherein determining the urgency level of the request is based upon a time remaining until the medical staff shift position needs to be filled.

7. The computerized process of claim 2, wherein publishing the request to a plurality of candidate staff members based upon the determined urgency level comprises:
when a time remaining for the medical staff shift position to be filled is greater than an urgent timing window, publishing the request through an staffing agency; and
when the time remaining for the medical staff position to be filled is less than an urgent timing window, publishing the request directly to the plurality of candidate staff members.

8. The computerized process of claim 2, wherein the computerized server device further operates programming configured to:
automatically identify open medical staff shift positions that need to be filled; and
automatically generate requests to fill the open medical staff shift positions.

9. The computerized process of claim 2, wherein the computerized server device further operates programming configured to:
if no responses are received to the published request, automatically increase pay associated with the request and re-publish the request.

10. The computerized process of claim 2, wherein the computerized server device further operates programming configured to:
monitor assignment of a case worker to the request; and
provide resources to the case worker to fill the request.

11. The computerized process of claim 10, wherein providing resources to the case worker comprises scheduling an appointment between the case worker and a patient named in the request.

12. The computerized process of claim 10, wherein providing resources to the case worker comprises enabling a patient named in the request to electronically sign a service contract for the request.

13. The computerized process of claim 2, wherein automatically select an elected staff member to fill the medical staff shift position from the monitored responses comprises:
assigning a score to each of the monitored responses; and
selecting the elected staff member based upon the assigned scores.

14. The computerized process of claim 13, wherein assigning the score to each of the monitored responses comprises evaluating the monitored responses based upon one of licensure, training, years of work experience, time worked in a particular role, and a worker specific pre-determined rating.

15. The computerized process of claim 2, wherein the computerized server device further operates programming configured to:
monitor the elected staff member clocking-in to work;
monitor the elected staff member clocking-out of work; and
automatically generating payment to the elected staff member based upon the monitored clocking-in and clocking-out.

16. The computerized process of claim 2, wherein the computerized server device further operates programming configured to request at least one of license certification and training certification from the candidate staff members.

17. The computerized process of claim 16, wherein the computerized server device further operates programming configured to automatically remind the candidate staff members regarding expiration dates of the license certification and the training certification.

18. A computerized process, comprising:
within a computerized server device, operating programming configured to:
monitor a job posting for a medical facility, the posting comprising at least one qualification selected from a licensing level, a training level, a pre-determined hospital specific rating level, and a pay level for the posting;
post a request to fill the job posting to a plurality of registered candidates;
receive responses from the plurality of registered candidates;
sorting the responses based upon the qualification of each of the candidates;
assigning one of the candidates to the job posting based upon the sorting; and
providing a report to the medical facility comprising the candidate assigned to the job posting.
